(19) United States
(12) Patent Application Publication
(10) Pub. No.: US 2017/0065577 A1
(43) Pub. Date: Mar. 9, 2017

(54) THERAPEUTIC COMPOSITIONS AND METHODS

(71) Applicant: UNIVERSITY OF IOWA RESEARCH FOUNDATION, Iwoa City, IA (US)

(72) Inventors: Edward A. Sander, Iowa City, IA (US); Aliasgar K. Salem, Iowa City, IA (US); James A. Martin, Iowa City, IA (US)

(73) Assignee: UNIVERSITY OF IOWA RESEARCH FOUNDATION, Iowa City, IA (US)

(21) Appl. No.: 15/260,100

(22) Filed: Sep. 8, 2016

Related U.S. Application Data
(60) Provisional application No. 62/215,594, filed on Sep. 8, 2015.

(51) Int. Cl. A61K 31/4745; A61K 9/00; A61K 47/34

(52) U.S. Cl. CPC A61K 31/4745; A61K 47/34; A61K 9/0014

(57) ABSTRACT

Therapeutic methods and compositions for treating fibrosis (e.g. scarring) are provided, as well as compositions comprising blebbistatin or a salt thereof and PLGA and nanoparticles comprising blebbistatin or a salt thereof and PLGA.

### RELATED APPLICATIONS

This application claims priority to U.S. Provisional Patent Application No. 62/215,594 filed Sep. 8, 2015, the entirety of which is incorporated herein by reference.

### GOVERNMENT FUNDING

This invention was made with government support under W81XWH-14-0327 awarded by the Department of Defense. The government has certain rights in the invention.

### BACKGROUND

Abnormal scars, such as hypertrophic scars, are characterized by excessive fibrosis that can result in disfigurement, distress, discomfort/pain, and permanent loss of function from contracture. Abnormal scarring is a major clinical problem with estimated U.S. annual treatment costs in the billions of dollars. There are a number of clinical treatments that have been explored to manage and reduce scarring but they have limited effectiveness. Furthermore, although much attention has been placed on controlling the chemical pathways that regulate scar formation and tissue fibrosis, few have investigated ways to alter the mechanical stimuli that also play a prominent role in fibrosis.

Currently there is a need for agents that are useful for treating or preventing scarring.

### SUMMARY

The present invention provides the drug blebbistatin to prevent abnormal scarring. In a particular embodiment of the invention, poly(lactic-co-gylcotic acid) (PLGA) nanoparticles comprising blebbistatin are used to deliver blebbistatin to cells in a wound site in order to prevent abnormal scarring. The drug reversibly interferes with the force generation/force sensing machinery of a cell and should mitigate the effect of tension and other mechanical cues in the wound site that would otherwise trigger a fibrotic healing response typified by excessive collagen production. Application of this drug/delivery system should reduce scar tissue and improve healing.

Accordingly, one embodiment provides a method to prevent or reduce scarring in a animal comprising administering a compound (e.g., blebbistatin or a salt thereof) that disrupts the force generating mechanism of a cell (e.g., a cell in a wound such as a wound on a mammal) to the animal (e.g., a human). In one embodiment the disruption is temporary (e.g., less than about 12 hrs, less than about 10 hrs or less than about 6 hrs). In one embodiment the disruption lasts greater than one day. In one embodiment the disruption lasts greater than two days. In one embodiment the disruption lasts greater than one week.

In one embodiment the invention provides a method to prevent or reduce scarring in a animal comprising administering a compound (e.g., blebbistatin or a salt thereof) that disrupts the force generating mechanism of a cell (e.g., a cell in a wound) to the animal.

In one embodiment the invention provides a method to prevent or reduce scarring in a animal comprising administering a compound (e.g., blebbistatin or a salt thereof) that disrupts the force generating mechanism of the cell (e.g., a cell in a wound) by blocking the activity of myosin II to the animal.

In one embodiment the invention provides a method to prevent or reduce scarring in a animal comprising administering a compound (e.g., blebbistatin or a salt thereof that blocks myosin II to a wound of the animal.

In one embodiment the invention provides a method to rapidly expand stem cells while maintaining their pluripotency, comprising contacting the stem cells with a composition comprising PLGA and blebbistatin or a salt thereof.

In one embodiment the invention provides a method to treat fibrosis in an animal (e.g., a mammal such as a human) comprising administering a compound (e.g., blebbistatin or a salt thereof) that blocks myosin II to the animal.

In one embodiment the invention provides a composition comprising PLGA and blebbistatin or a salt thereof.

In one embodiment the invention provides a composition comprising PLGA nanoparticles and blebbistatin or a salt thereof.

In one embodiment the invention provides a nanoparticle comprising PLGA and blebbistatin or a salt thereof.

In one embodiment the invention provides a polymer-based particle (e.g., a controlled release polymer-based particle) comprising PLGA and blebbistatin or a salt thereof.

In one embodiment the invention provides a compound (e.g., blebbistatin or a salt thereof) that blocks myosin II for the prophylactic or therapeutic treatment of scarring.

In one embodiment the invention provides a nanoparticle comprising PLGA and blebbistatin or a salt thereof for the prophylactic or therapeutic treatment of scarring.

In one embodiment the invention provides the use of a compound (e.g., blebbistatin or a salt thereof) that blocks myosin II for the preparation of a medicament for the prophylactic or therapeutic treatment of scarring.

In one embodiment the invention provides the use of PLGA and blebbistatin or a salt thereof to prepare a medicament for the prophylactic or therapeutic treatment of scarring.

In one embodiment the invention provides the use of a nanoparticle comprising PLGA and blebbistatin or a salt thereof to prepare a medicament for the prophylactic or therapeutic treatment of scarring.

### BRIEF DESCRIPTION OF THE FIGURES

FIG. 1 shows a scanning electron microscope image demonstrating the topography and size distribution of PLGA nanoparticles. The particles shown span a size range of 200 nm to 2 μm.

FIG. 2 shows a graph demonstrating the release profile of blebbistatin from the PLGA nanoparticles over a 7-day period (168 hours). The y-axis indicates the % of total drug cumulatively released from the particles for three separate experiments with data points and error bars indicating mean and standard deviation, respectively. This plot indicates that for this particular formulation of PLGA particles, 70% of the blebbistatin is released within 10 hours.

FIG. 3 shows the change in area over time of collagen gel uniformly seeded with rabbit joint capsule fibroblasts and subjected to different treatments. The change in gel area is caused by the embedded cells using force to compact the gel. Untreated gels compacted the most, reducing to 42%±2% of the initial area. These gels served as controls and provide a baseline for how much force the resident cells generate. Blank PLGA particles did not alter the amount of compaction observed in untreated gels (44%±1% of initial area). Blebbistatin PLGA particles significantly limited compaction (82%±2% of initial area) indicating that the drug was effective in blocking myosin II and actin interactions necessary to generate force in a manner that was nearly as effective as administration of blebbistatin solubilized in DMSO (87%±1% of initial area) and added directly to the gels without the PLGA particles. The same concentration of DMSO alone also reduced the amount of compaction in the gel but not nearly to the same extent as treatment with blebbistatin did. (53%+1% of initial area)

FIGS. 4A-C show Rho/ROCK pathway gene expression analysis using qPCR on RNA isolated from rabbit joint capsule fibroblasts derived from cultures at respective time points with indicated treatments. (FIG. 4A) RhoA expression (n=3). (FIG. 4B) ROCK1 expression (n=3). (FIG. 4C) ROCK2 gene expression (n=3). Values are expressed as mean±SD. Rho/ROCK is a critical signaling pathway involved in cell force sensing and force generation. The data show an initial increase in RhoA, ROCKI, and ROCK II genes treated with blebbistatin that then decrease to comparable levels as control. This temporal effect is consistent with the release profile of the drug.

FIGS. 5A-C shows (FIG. 5A) less gel compaction in blank particle control gels compared to blebbistatin PLGA particle gels; (FIG. 5B) microscopic images of individual rabbit joint capsule fibroblasts with embedded inert microsphere in these gels for tracking local displacements as an indicator of cell force generation; and (FIG. 5C) quantification of these displacements over the duration of the experiment show much larger displacements in the control gel and that the drug in the dosage used temporarily stops cell force generation.

### DETAILED DESCRIPTION

Upon injury, the body initiates a wound-healing process to restore homeostasis. There are three overlapping phases involved in this process: inflammation, proliferation, and remodeling. In the inflammation phase, neutrophils and other leukocytes infiltrate the provisional fibrin matrix filling the wound site, mitigate foreign agents, and release chemicals to recruit fibroblasts, endothelial cells, and other cell types to the wound site. In the proliferation phase, fibroblasts and other cells differentiate into myofibroblasts and deposit extracellular matrix (ECM) proteins (e.g. collagen, proteoglycans, and attachment proteins) to form a new ECM. In the remodeling phase, apoptosis eliminates myofibroblasts and extraneous blood vessels, and the ECM is remodeled to resemble the original tissue. During the healing process, elevated mechanical forces can trigger abnormal scar formation via the excessive production of collagen and other ECM proteins by the resident myofibroblasts. Reducing the magnitude of these forces can reduce collagen production, thus leading to normal healing.

### Polymer-Based Particles Including Nanoparticles

Polymer-based particles including controlled release polymer-based particle includes particles of any functional size to allow for the delivery of chemicals (e.g., therapeutic agents) to tissues of the body. In one embodiment the polymer-based particles (e.g., polymer-based particles such as controlled release polymer-based particles and PLGA particles) are about 0.1 nm to 1000 um in diameter. In one embodiment the polymer-based particles (e.g., controlled release polymer-based particles such as PLGA particles) spanning a range of 0.1 nm to 1000 μm are administered at the site of a wound to prevent or reduce scarring. Other examples of controlled release polymer-based particles, include but are not limited to poly(ε-caprolactone), dextran, and lipid nanoparticles. For additional information see Journal of Controlled Release, 161, 505-522 (2012) and Biomaterials, 32, 9826-9838 (2011).

Compositions provided herein and compounds of the methods provided herein can be nanoparticles and/or administered as nanoparticles. In one embodiment the polymer-based particles (e.g., controlled release polymer-based particles such as PLGA particles) are nanoparticles. In one embodiment the nanoparticles are less than or about 10 μm in diameter. In one embodiment the nanoparticles are less than or about 1 μm in diameter. In one embodiment the nanoparticles are less than or about 750 nm in diameter. In one embodiment the nanoparticles are less than or about 250 nm in diameter. In one embodiment the nanoparticles are about 0.1 nm to about 1 μm in diameter. In one embodiment the nanoparticles are about 1 nm to about 1 μm in diameter. In one embodiment the nanoparticles are about 1 nm to about 750 nm in diameter.

### Blebbistatin

Blebbistatin (674289-55-5) is a selective inhibitor of non-muscle myosin II ATPase activity. Blebbistatin is commercially available. It reversibly binds to inhibit myosin II, preventing myosin II from activating actin, thereby preventing actin from producing tension. Fibroblasts exposed to blebbistatin should exhibit diminished mechanosensitivity and respond by reducing collagen production. Normally blebbistatin is formulated with or carried in dimethyl sulfoxide (DMSO), however there is currently a controversy regarding whether DMSO is toxic. Accordingly, in one embodiment the invention provides compositions and nanoparticles that comprise blebbistatin or a salt thereof. The PLGA containing compositions and nanoparticles allow for the delivery of blebbistatin or a salt thereof without DMSO.

### PLGA

PLGA is a lactic acid and glycolic acid co-polymer that is commercially available. It has been studied as a delivery mechanism (carrier) for small molecule drugs, proteins, and other macromolecules. PLGA is biocompatible and biodegradable, exhibits a wide range of erosion times, has tunable mechanical properties, provides the possibility to target nanoparticles to specific organs or cells, is FDA approved, and has minimal systemic toxicity.

Useful dosages of the myosin II blocker can be determined by comparing their in vitro activity, and in vivo activity in animal models. Methods for the extrapolation of effective dosages in mice, and other animals, to humans are known to the art; for example, see U.S. Pat. No. 4,938,949.

The amount of the myosin II blocker, or an active salt or derivative thereof, required for use in treatment will vary not only with the particular salt selected but also with the route of administration, the nature of the condition being treated and the age and condition of the patient and will be ultimately at the discretion of the attendant physician or clinician.

### EMBODIMENTS

The ability of a myosin II blocker to reduce scarring can be determined using pharmacological models which are well known to the art, or using Test A described below.

### Test A

In vitro quantification of the efficacy of a treatment such as the administration of blebbistatin PLGA particles, on collagen production can be done by quantifying the amount of collagen produced over a period of time by the cells embedded in a fibrin gel. In the assay, gel is treated with blebbistatin PLGA particles or blank PLGA particles. Fibrin is used as a simple model of the initial wound site, as fibrin is the main structural component of a clot. The gels are maintained in cell culture medium in a 5%/95% CO₂/O₂ incubator maintained at 37° C. After 7 to 14 days of culture, the gels are assayed for cell number and hydroxyproline content. The amount of hydroxyproline in the gel can be directly correlated to the amount of collagen produced by the cells. The expectation is that lower collagen production with the treatment, in this example the application of blebbistatin PLGA particles, compared to controls in this assay will translate to reduced scarring and fibrosis at the wound site.

The next step is the evaluation of treatment effect on scarring in a porcine models of wound healing: Pigs are the preferred animal model for demonstrating the efficacy of a wound healing treatment, as the wound healing response in this animal most closely resembles that in humans (Int Wound J. 2013 June; 10(3):295-305. doi: 10.1111/j.1742-481X.2012.00976.x. Epub 2012 May 8. Experimental pig model of clinically relevant wound healing delay by intrinsic factors. Jung, Y., et al.; Wound Repair Regen. 2001 March-April; 9(2):66-76, The pig as a model for human wound healing, Sullivan T. P., et al.; Ann Surg. 2011 August; 254(2):217-25. doi: 10.1097/SLA.0b013e318220b159. Improving cutaneous scar formation by controlling the mechanical environment: large animal and phase I studies. Gurtner, G. C., et al.). Based on the dosing determined with the in vitro assay, the extent of scarring on excisional wounds treated with controlled-release polymer-based particles can be compared to contralateral controls treated with blank particles several weeks (e.g., 8 weeks) after injury.

The invention will now be illustrated by the following non-limiting Examples.

### Example 1

### PLGA Particle Preparation

PLGA nanoparticles were produced using an oil in water (0/W) single emulsion technique. The method requires preparation of an organic phase and an aqueous phase. The organic phase was prepared by dissolving 3 mg of blebbistatin in 1.5 mL of dichloromethane (DCM) to which 200 mg of medical grade PLGA (Resomer® RG 503; Boehringer Ingelheim KG, Germany) was added and vortexed until the polymer dissolved completely. The aqueous phase was prepared by using polyvinyl alcohol (PVA; Mowiol®; Sigma, Allentown, Pa.) as a stabilizer so that the final concentration of PVA was 1%. The single emulsion was produced by the dropwise addition of 1.5 mL of the organic phase to 8 mL of the aqueous phase followed by sonication with a Sonic Dismembrator (Model FB 120 equipped with an ultrasonic converter probe CL-18; Fisher Scientific, Pittsburgh, Pa.) at 40% amplitude for 30 seconds. The resulting emulsion was then added to 22 mL of 1% PVA to induce diffusion of the organic phase into the continuous phase and was stirred under a chemical fume hood using a magnetic stirrer for 2 hours in order to evaporate the DCM. The resulting emulsion was then centrifuged with an Eppendorf Centrifuge 5804 R (Eppendorf, Westbury, N.Y.) at 1500 g for 10 minutes to collect the suspended particles. The supernatant was discarded and the resulting pellet was resuspended in 30 mL of ultrapure water and centrifuged again. This process was repeated twice to wash out any remnants of the solvent (DCM) left attached to the particles. The pellet was resuspended in 3 mL of ultrapure water, frozen at −20° C. for 24 hours, and then lyophilized at a collector temperature of −53° C. and 0.08 mBar pressure for 24 hours using a LABCONCO freeze dry system (FreeZone® 4.5 l, Model 7750020; Labconco Corporation, Kansas City, Mo.). The weight of the lyophilized product was determined and used to calculate entrapment efficiency and drug loading (defined below, c.f. XX). Blank PLGA nanoparticles without blebbistatin were also prepared in an identical manner as a control.

### Physicochemical Characterization of Blebbistatin Loaded Nanoparticles

### Scanning Electron Microscopy

Scanning electron microscopy (SEM) was employed to characterize particle size and morphology. In brief, the suspension of particles was diluted with ultrapure water at the ratio of 1 to 10 in order to limit particle agglomerates. Drops of this suspension were then placed on silicon wafers, each of which was then mounted onto a SEM stub and allowed to dry for 24 hours. The stubs were coated with a layer of gold-palladium by an argon beam K550 sputter coater (Emitech Ltd., Kent, England) and imaged with a Hitachi S-4800 Field-Emission SEM (Hitachi Ltd., Tokyo, Japan). Images were captured at 4 kV accelerating voltage under an argon atmosphere. Particle size was assessed by importing images into ImageJ (US National Institutes of Health, Bethesda, Md., USA) for analysis.

### Raman Spectroscopy

Confocal Raman spectroscopy was used to characterize the distribution of blebbistatin in the PLGA nanoparticles. Spectra of pure blebbistatin, pure PLGA, blank nanoparticles, and blebbistatin loaded nanoparticles were produced with the 488 nm line of an Ar+ laser (Coherent, Inova 70, 30 mW). After autoalignment of the instrument, the laser beam was focused on the samples using an inverted optical microscope equipped with a 50× objective lens. Spectra were recorded between 80 cm⁻¹ to 1300 cm⁻¹ at high resolution. Spectra were spatially resolved at each pixel by passing the backscattered image of the Ar+ laser through a pinhole of diameter 100 μm onto a cooled CCD camera.

### Differential Scanning Calorimetry for Drug Solid State Analysis in PLGA Particles

Differential scanning calorimetry was employed to analyze the solid state and thermal properties of the nanoparticles. Approximately 2 mg of either lyophilized blank PLGA nanoparticles or drug-loaded PLGA nanoparticles were weighed and hermetically sealed into aluminum pans. An empty aluminum pan was used as a reference. Sample were heated from 25° C. to 300° C. at a rate of 10° C./min under a stream of nitrogen gas flow to produce thermograms for extracting the transition temperature (Tc) and enthalpy of transition (ΔH).

### HPLC Method for In Vitro Studies

The concentration of blebbistatin was measured via high pressure liquid chromatography (HPLS) with a Waters HPLC system with a UV detector (Waters 484) and Waters xyz autosampler. The solvents were pumped through a Luna C₁₈ column (250 mm×10 mm) with isocratic flow. The mobile phase was comprised of water (0.15% triethanolamine) and acetonitrile (0.15% triethanolamine) at a ratio of 3:7 (v/v). Before pumping the mobile phase through the HPLC column, the mobile phase was filtered using a 0.22 μm Millipore membrane filter and sonicated for 30 minutes. The HPLC column was washed initially with a methanol and water mobile phase (75:25), and then equilibrated with the degassed mobile phase (water:ACN 3:7). The samples were loaded in respective stations. The injection volume was 10 μL and the detection wavelength was 425 nm. The mobile phase flow rate was maintained at 0.5 mL/min and the run time was set to 5 min. All the data obtained were collected and analyzed in Empower Pro Chromatography Manager Data Collection System. Calibration curve measurements were performed using 6 different concentration standards for the determination of blebbistatin entrapment efficiency and release profile.

### Drug Entrapment Efficiency and Nanoparticle Yield Determination

The entrapment efficiency of blebbistatin in PLGA nanoparticles was determined by taking the ratio of the amount of blebbistatin entrapped in the particles to the total amount of drug added. Briefly, 10 mg of drug loaded nanoparticles was dissolved in 5 mL of acetonitrile and stored for 30 minutes in an oven set to 40° C. The suspension was then sonicated for 30 minutes and centrifuged at 5000 g for 10 minutes. The resulting supernatant was collected and filtered with a 0.45 μm filter. Absorbance of the supernatant was measured with HPLC. The measured absorbance was converted to the amount of entrapped drug via a calibration curve.

Entrapment Efficiency %=(Experimental drug content/Total drug content)×100

Nanoparticle Yield %=(Mass of drug loaded nanoparticles obtained/Initial weight of polymer+drug)×100

### Release Study of Blebbistatin

A quantitative release study was performed in phosphate-buffered saline (PBS) at pH 7.4 with 1% Tween 80 in an incubator shaker set at 37° C. and 300 rpm. Approximately, 20 mg of blebbistatin loaded nanoparticles was weighed and transferred into a 50 mL conical tube to which 20 mL of PBS with 1% Tween 80 was added and mixed well so that the final concentration was 1 mg/mL. The resulting nanoparticle suspension was then divided into 20 eppendorf tubes so that each tube had 1 mL of 1 mg/mL nanoparticle suspension. All tubes were labeled, and at the corresponding time intervals (0.5, 1, 2, 3, 4, 5, 6, 7, 8, 9, 10, 24, 48, 72, 96, 120, 144 and 168 h) the tubes were withdrawn and centrifuged at 5000 g for 10 min. The resulting supernatant was then filtered through a 0.45 μm filter and analyzed for absorbance using HPLC/UV detection as mentioned above.

### In Vitro Studies of Blebbistatin Loaded Nanoparticle Activity on Rabbit Joint Capsule Fibroblasts

### Collagen Gel Compaction Assay

The effect of blebbistatin on rabbit joint capsule fibroblasts (RJCF) was analyzed using a collagen gel compaction assay. RJCFs between passage 4 and 9 were trypsinized using 0.1% trypsin/EDTA for 5 minutes at 37° C. and then the added trypsin was neutralized using DMEM containing 10% FBS. The cell suspension was centrifuged at 230 g for 5 minutes and resuspended in a desired volume of 10% FBS containing DMEM. The collagen gels loaded with RJCFs were prepared by mixing 10×PBS containing phenol red, 1N NaOH, rat tail collagen-I, and the cell suspension to give a final yield of 1×10⁶ cells/mL and 2 mg/mL collagen. 500 μL of the collagen gel mixture was added to each well in a 24 well plate and allowed to gelate for 30 minutes in an incubator. After 30 minutes 500 μL of DMEM containing 10% FBS and 50 μg/mL ascorbic acid was added to each well and incubated for 24 hours. After 24 hours, 2.5 mg of blank and blebbistatin loaded nanoparticles dispersed in 50 μL of DMEM or 50 μL of pure drug dissolved in DMSO (1 mg/mL) or 50 μL DMSO or 50 μL PBS were added to each gel so that the final blebbistatin concentration was 50 μg/mL or a corresponding solvent volume for controls. The gels were divided into five groups: (1) blebbistatin PLGA particles served as treatments, (2) pure drug dissolved in DMSO (1 mg/mL) served as a positive control, (3) blank PLGA particles, (4) DMSO alone, and (5) untreated gels served as negative controls. Each group was maintained under their respective conditions for 24 hours, before being washed twice with fresh medium. The gels were then dislodged from the well plate using a sterile spatula. Images were acquired at 0, 0.15, 0.5, 1, 2, 3, 4, 5, 6, 7, 24 and 48 hours, and the amount of compaction was measured with ImageJ.

### Real Time PCR Analysis

Inhibition of Rho/ROCK pathway in RJCFs by blebbistatin nanoparticle was studied using quantitative real-time PCR (qPCR) Two groups of collagen gels were prepared in the same way as described in the collagen gel compaction assay. One group of gels (n=3) was treated with 2.5 mg of blebbistatin nanoparticles dispersed in 50 μL of DMEM so that the final concentration of blebbistatin was 50 μg/mL. The other control group gels (n=3) were also treated in the same way but instead with blank PLGA particles. Measurements were made at four terminal time points of 0, 5, 24 and 48 hours. After each time point the gels were washed twice with DMEM and stored at −80° C. for qPCR analysis.

Total cell RNA was extracted with an RNeasy Mini Kit (Qiagen, Valencia, Calif., USA), by following the manufacturer's protocol. Cells were then homogenized (QIAshredder column, Qiagen), and using an RNeasy column, total RNA was eluted out. The quantity and the quality of the isolated RNA were measured from the absorbance at A260 nm and the ratio of A260/A280 respectively. Reverse transcription reactions were carried out on the extracted RNA with TaqMan Reverse Transcription Reagents (Applied Biosystems, Foster City, Calif., USA), and the reverse transcription reactions were carried out in a PTC-200 Peltier Thermal Cycler (MJ Research, BioRad, Walthan, Mass., USA). Initially the mixture was subjected to 25° C. for 10 min, then incubated at 48° C. for 30 min, and this mixture was heated at 95° C. for 5 min, and finally chilled to 4° C. TaqMan Ribosomal RNA Control Reagents Kit (Applied Biosystems, Foster City, Calif., USA) was used to detect 18 s rRNA as an endogenous control. TaqMan Universal PCR Master Mix (Applied Biosystems), primers and probes for Rho, ROCK1, ROCK2, and the endogenous 18 s rRNA control, and the cDNA were mixed in 96-well Optical Reaction Plates (Applied Biosystems), and qPCR reactions were performed in an Applied Biosystems 7300 Real Time PCR System, with thermal cycling parameters set at 50° C. for 2 min, 95° C. for 10 min, 40 cycles of 95° C. for 15 s, and 60° C. for 1 min. Steady-state mRNA levels were normalized to 18 s rRNA and calculated relative to untreated controls by “the relative quantitation using comparative C_{T}” in Multiplex Reactions (PerkinElmer).

### Fluorescent Microsphere Displacement Assay

The effect of blebbistatin on cell ability to compact gels was analyzed by monitoring the displacement of fluorescent microspheres. Cell seeded collagen gels were prepared as described above with the addition of a suspension of fluorescent 4 μm microspheres to achieve a concentration of 5 million microspheres/mL. Approximately, 500 μL of this solution were then carefully added around a 6 mm polydimethysiloxane (PDMS) post in a circular PDMS mold with a 20 mm diameter attached to a 35 mm glass bottom Petri dish. This construct was prepared with the purpose of preventing movement of the gel during imaging, after release. Gels were then incubated at 37° C. for 30 minutes to allow polymerization. Once the gels had polymerized, DMEM supplemented with 10% FBS, 1% Penicillin/Streptomyocin, and 0.1% Amphoterecin B was added. After incubation for 24 hours, the gels were treated with either blebbistatin PLGA particles or with blank PLGA particles for 24 hours. Gels were carefully detached from the surrounding PDMS mold and were rinsed twice using DMEM. A fresh volume of DMEM was added to the samples which were then transferred onto the stage of a Nikon Eclipse Ti microscope, enclosed by an environmental chamber to maintain temperature at 37° C., and a gas phase of 5% CO₂ Each sample was then imaged at four different locations using DIC and epifluorescent imaging. Microsphere displacements were quantified by use of a custom algorithm using digital image correlation to track the movement of individual particles from frame to frame.

### Results

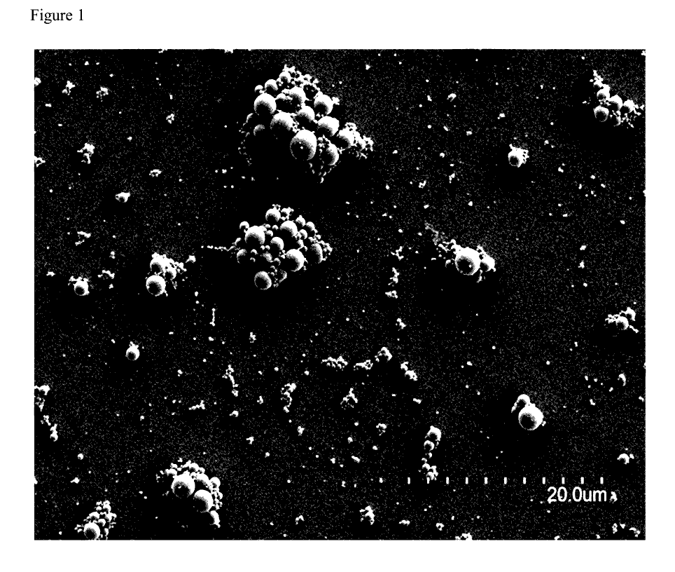

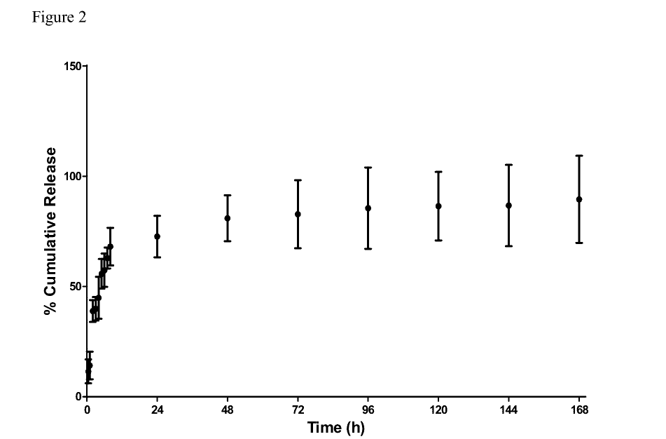

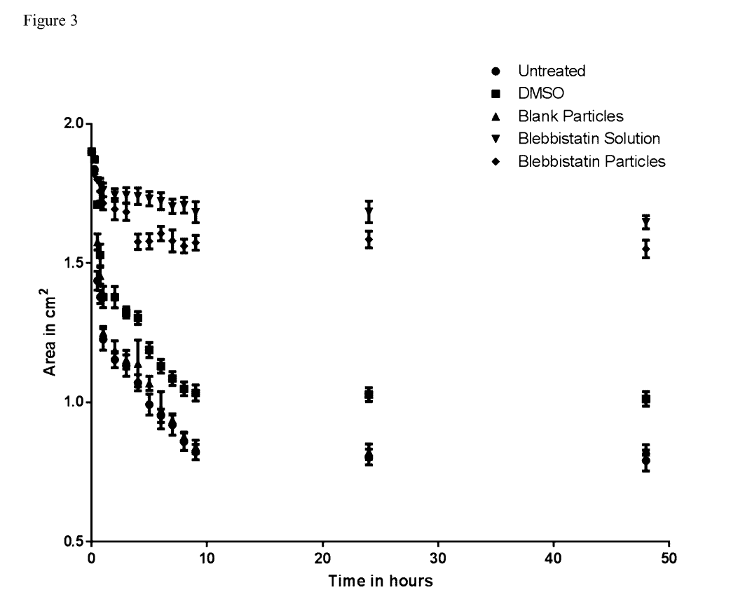

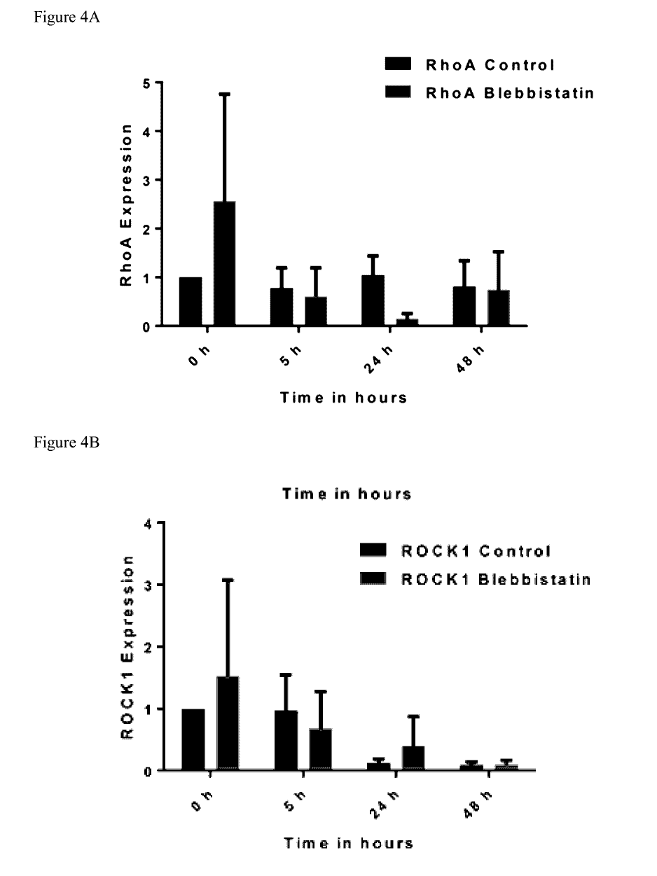

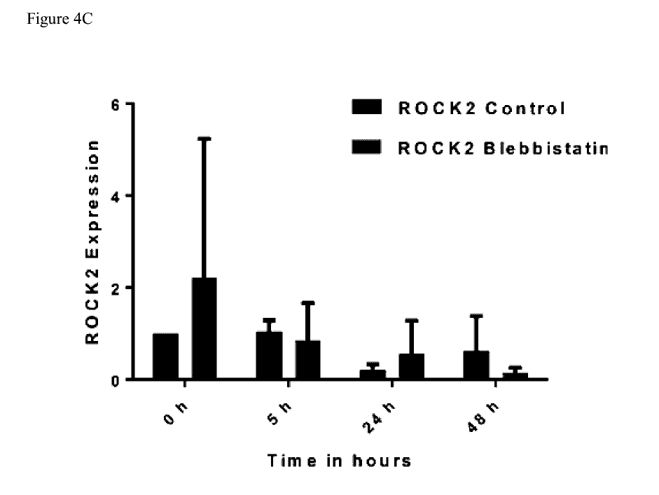

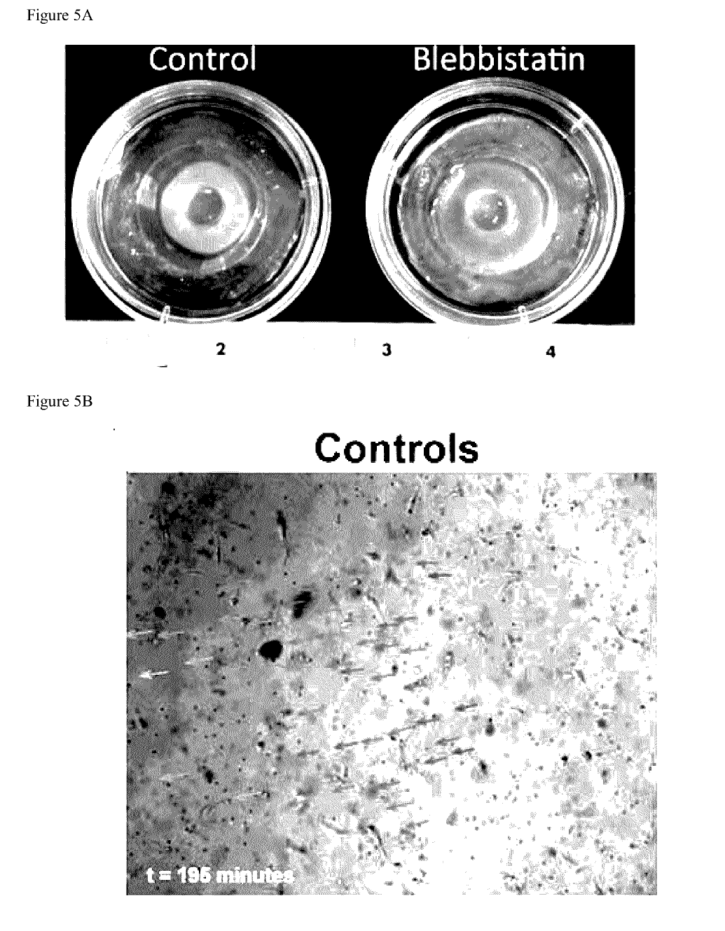

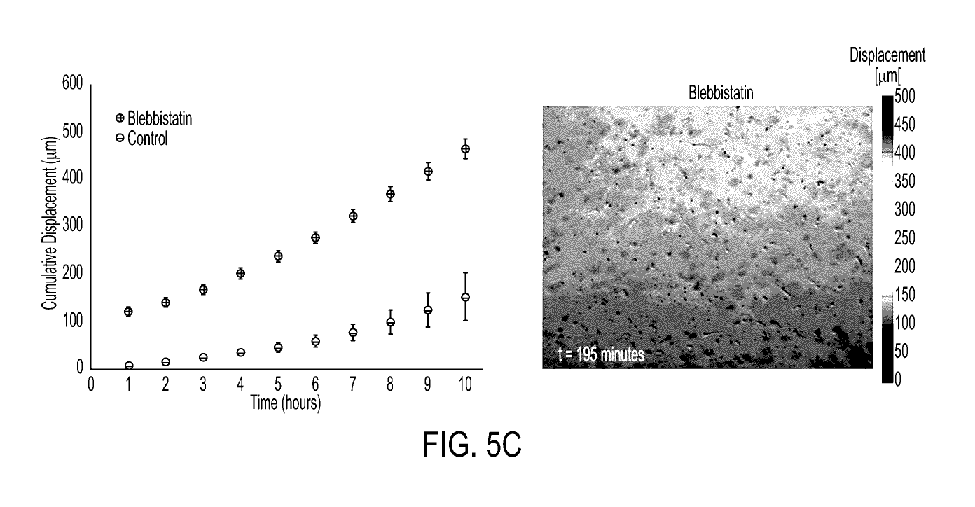

SEM images demonstrated that the size of the particles ranged between 0.2 μm to 5 μm. (FIG. 1) Raman spectroscopy results demonstrated that the significant Raman shift wavenumbers of blebbistatin at around 1420 cm⁻¹, 1280 cm⁻¹ and 810 cm⁻¹ were not changed due to the loading of blebbistatin. DSC results demonstrated that the melting point of blebbistatin at 204° C. was also unaffected due to loading of drug into the particles, indicating that the drug is stable and no significant physical/chemical changes occurred. Encapsulation efficiency of the drug was found to be approximately 65%, and the cumulative release profile showed an initial burst release until 10 hours followed by a sustained release (FIG. 2). Fibroblast-seeded collagen gels treated with blebbistatin nanoparticles only slightly decreased in surface area, whereas the untreated gels and the other control gels decreased approximately 40-50% in surface area, indicating that the drug did indeed reduce substantially fibroblast force generation (FIG. 3). Furthermore, blebbistatin nanoparticles were as effective in limiting gel compaction as DMSO-solubilized blebbistatin directly added to the media was. qPCR data indicate that RhoA, ROCKI, and ROCK II gene expression initially increased when treated with blebbistatin PLGA particles that then decreased to comparable levels as the control. This temporal effect is consistent with the release profile of the drug. Time-lapse imaging revealed significant changes in fibroblast morphology, with fibroblasts in untreated gels appearing spindle shaped and branched compared to more spherical fibroblasts in blebbistatin gels. Fluorescent microsphere displacements were also significantly lower in blebbistatin treated gels compared to untreated collagen gels.

All publications (including Atluri, K., et al., ACS Biomater. Sci. Eng., 2016, 2, 1097-1107) patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A method to prevent or reduce scarring in an animal comprising administering a compound that disrupts the force generating mechanism of a cell to the animal.

2. The method of claim 1 wherein the compound disrupts the force generating mechanism of the cell by blocking the activity of myosin II.

3. The method of claim 1 where the compound blocks interactions between myecin and actin.

4. The method of claim 1, wherein the disruption is temporary.

5. The method of claim 1, wherein the compound is blebbistatin or a salt thereof.

6. The method of claim 1, wherein the cell is in a wound.

7. The method of claim 6, wherein the wound is on a mammal.

8. The method of claim 1, wherein a composition comprising PLGA and blebbistatin or a salt thereof is administered to the wound.

9. The method of 1, wherein a composition comprising PLGA nanoparticles and blebbistatin or a salt thereof is administered to the wound.

10. A method to treat fibrosis in an animal comprising administering a compound that blocks myosin II to the animal.

11. The method of claim 10 wherein the compound is blebbistatin or a salt thereof.

12. The method of claim 10 wherein the compound is administered topically to the animal.

13. The method of claim 10 wherein the animal is a human.

14. The method of claim 10 wherein a composition comprising PLGA and blebbistatin or a salt thereof is administered.

15. The method of claim 10 wherein a composition comprising PLGA nanoparticles and blebbistatin or a salt thereof is administered.

16. A method to rapidly expand stem cells while maintaining their pluripotency, comprising contacting the stem cells with a composition comprising PLGA and blebbistatin or a salt thereof.

17. A composition comprising PLGA and blebbistatin or a salt thereof.

18. The composition of claim 17 which comprises PLGA nanoparticles and blebbistatin or a salt thereof.

19. A nanoparticle comprising PLGA and blebbistatin or a salt thereof.
